(19) United States
(12) Patent Application Publication
(10) Pub. No.: US 2013/0190860 A1
(43) Pub. Date: Jul. 25, 2013

(54) PERCUTANEOUSLY DELIVERABLE VALVES

(71) Applicant: Thoralf M. Sundt, III, Boston, MA (US)

(72) Inventor: Thoralf M. Sundt, III, Boston, MA (US)

(73) Assignee: MAYO FOUNDATION FOR MEDICAL EDUCATION AND RESEARCH, Rochester, MN (US)

(21) Appl. No.: 13/704,197

(22) PCT Filed: Jun. 15, 2011
(86) PCT No.: PCT/US11/40484

§ 371 Date: Apr. 8, 2013

Related U.S. Application Data
(60) Provisional application No. 61/354,812, filed on Jun. 15, 2010.

(51) Int. Cl. A61F 2/24

(52) U.S. Cl. CPC A61F 2/2418
USPC 623/2.13; 623/2.17; 623/2.18

(57) ABSTRACT

This document provides methods and materials related to providing a mammal with a replacement valve (e.g., a synthetic or artificial heart valve). For example, synthetic or artificial heart valve that can be delivered in a minimally invasive manner are provided.

### CROSS-REFERENCE TO RELATED APPLICATIONS

This application claims the benefit of U.S. Provisional Application Ser. No. 61/354,812, filed Jun. 15, 2010. The disclosure of the prior application is considered part of (and is incorporated by reference in) the disclosure of this application.

### TECHNICAL FIELD

This document relates to synthetic valves that can be delivered in a minimally invasive manner.

### BACKGROUND

Heart valves are important components of a heart that allow the heart to function normally. In general, natural heart valves can allow for unidirectional blood flow from one chamber of the heart to another. In some cases, natural heart valves can become dysfunctional to a degree that may require complete surgical replacement of the natural heart valve with a heart valve prostheses.

### SUMMARY

This document provides methods and materials related to providing a mammal with a replacement valve (e.g., a synthetic or artificial heart valve). For example, this document provides synthetic or artificial heart valve that can be delivered in a minimally invasive manner.

In general, one aspect of this document features an artificial heart valve for placement within a mammal. The heart valve comprises, or consists essentially of, (a) at least two struts having a proximal portion and a distal portion, wherein the proximal portion is configured to attach to heart tissue, and wherein the struts, when the heart valve is placed within the mammal, converge towards an axis in a direction from the proximal portion to the distal portion, and (b) a membrane structure attached to the struts and configured to form a wall around the axis, wherein at least a portion of the membrane structure is capable of expanding and collapsing movement, wherein during the expanding movement the portion of the membrane structure moves away from the axis to form a closed position of the heart valve, wherein during the collapsing movement the portion of the membrane structure moves toward the axis to form an opened position of the heart valve, wherein, when the heart valve is placed within the mammal and in the opened position, blood upstream of the heart valve is capable of moving past the heart valve between the membrane structure and the mammal's heart tissue, and wherein, when the heart valve is placed within the mammal and in the closed position, movement of blood upstream of the heart valve past the heart valve between the membrane structure and the mammal's heart tissue is limited. The mammal can be a human. The proximal portion of the struts can be configured to attach to heart tissue via an adhesive, clamp, staple, barb, suture, hook, screw, or combination thereof. The membrane structure can comprise flexible biocompatible material. The membrane structure can comprise a polymer. The membrane structure can comprise animal pericardium tissue. The membrane structure can form a conical shape. The membrane structure can form a conical shape defining a lumen comprising an opening at first end and an opening at a second end, wherein the opening at the first end is larger than the opening at the second end. The heart valve can comprise a first end defining a diameter and a second end defining a diameter, wherein the diameter of the first end is larger than the diameter of the second end, and wherein the first end defines an opening. The second end can define an opening, wherein the opening of the second is smaller than the opening at the first end. The struts can comprise flexible material. The struts can comprise a shape memory material. The shape memory material can be nitinol. The heart valve can be capable of being placed within the mammal percutaneously. The heart valve can be capable of moving from a collapsed position during delivery to the mammal to an expanded position after placement within the mammal. The heart valve can comprise a ring structure attached to the proximal portion of the struts. When the heart valve is placed within the mammal and in the opened position, blood upstream of the heart valve can be capable of moving past the heart valve between the membrane structure and the ring structure, and when the heart valve is placed within the mammal and in the closed position, movement of blood upstream of the heart valve past the heart valve between the membrane structure and the ring structure can be limited. The ring structure can comprise a shape memory material biased to promote movement of the proximal portion of the struts away from the axis during placement of the heart valve within the mammal. The heart valve can comprise a ring structure attached to the distal portion of the struts. The heart valve can comprise a tethering cord anchor. The tethering cord anchor can be configured to extend from the heart valve and across at least a portion of the heart chamber downstream of the heart valve when the heart valve is placed within the mammal. The tethering cord anchor can comprise an adhesive, clamp, staple, barb, suture, hook, screw, or combination thereof configured to attach the tethering cord anchor to a wall of the heart chamber.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIGS. 1A-1B depict a replacement valve configured for minimally invasive delivery to the heart, wherein a membrane material that covers strut supports can collapse around those supports, in accordance with some embodiments.

FIG. 2 depicts the valve of FIGS. 1A-1B deployed in the aortic position, thus replacing the native aortic valve, in accordance with some embodiments.

FIGS. 3A-3B depict catheter deployment of the valve of FIGS. 1A-1B from a transapical approach, in accordance with some embodiments.

FIGS. 4A-4C depict multiple embodiments of a replacement valve.

FIGS. 5A-5E depict multiple embodiments of a replacement valve.

FIG. 6 depicts a replacement valve positioned at the mitral position, in accordance with some embodiments.

FIG. 7 depicts a replacement valve, including cord anchors, positioned in the aortic position, in accordance with some embodiments.

FIG. 8 depicts a replacement valve, including artificial chordae anchors, in accordance with some embodiments.

FIG. 9 depicts an annuloplasty ring positioned in a heart, in accordance with some embodiments.

FIG. 10 depicts a replacement valve including a two piece design, in accordance with some embodiments.

FIG. 11 depicts a replacement valve, in accordance with some embodiments.

FIG. 12 depicts a system for removing native valve tissue and deploying a replacement valve, in accordance with some embodiments. Like reference symbols in the various drawings indicate like elements.

### DETAILED DESCRIPTION

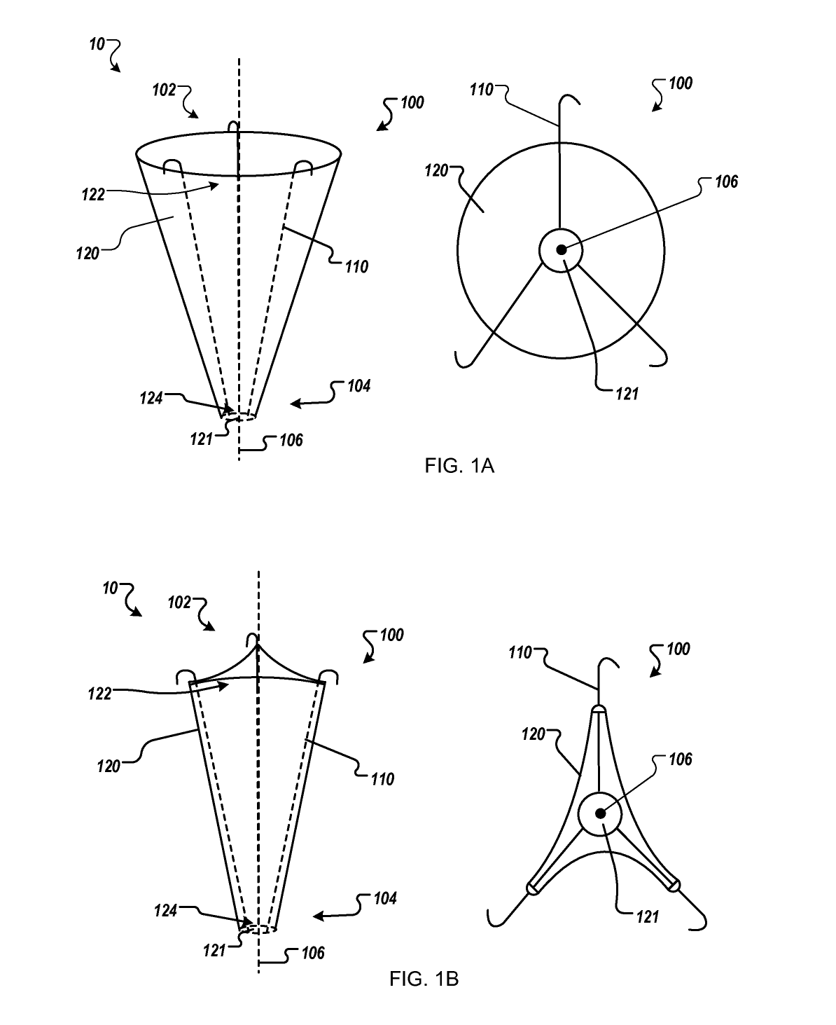

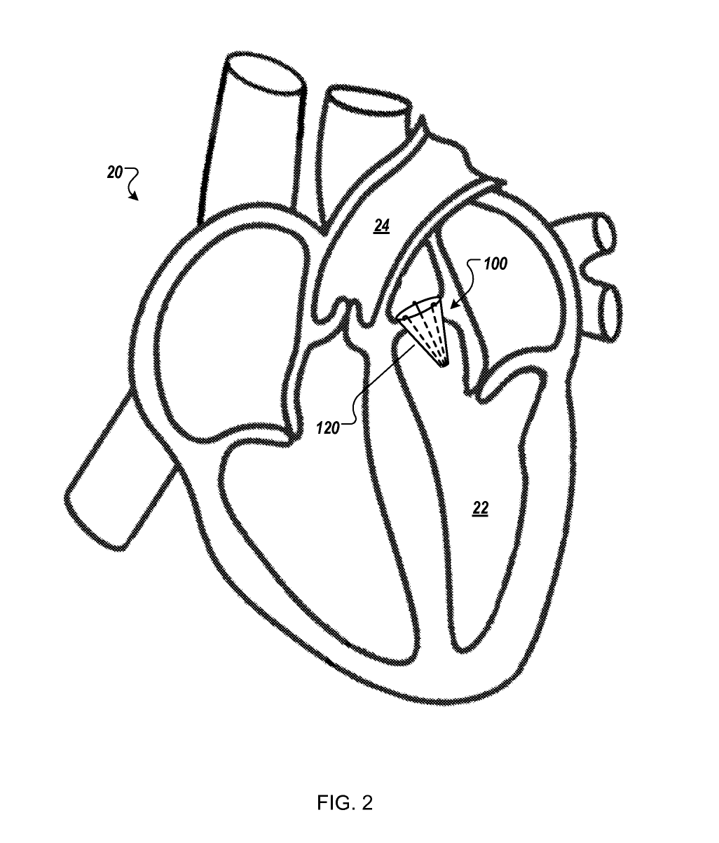

Referring now to FIGS. 1A and 1B, in some embodiments, a minimally invasive valve replacement system 10 includes an implantable valve 100 that further includes collapsible/expandable support struts 110 and a flexible membrane 120 covering the struts 110. In some embodiments, the implantable valve 100 can include three struts 110 covered by the pliable membrane 120 such that the struts 110 are able to engage the annulus of a native valve. In some cases, the struts can have anchoring devices that can embed into the native tissue. For example, a system provided herein can include self-fixing struts (e.g., struts with hooks or barbs along with helices). The struts 110 can form a generally conical shape, and the membrane 120 can include an opening 121 at the tip of the cone to prevent blood from pooling within the valve body. Once the valve 100 is deployed, the struts 110 can remain in a fixed position while portions of the membrane 120 can move radially in and out over the circumference of the valve 100 to facilitate the passage of fluids in one direction, while preventing or minimizing fluid movement in the opposite direction.

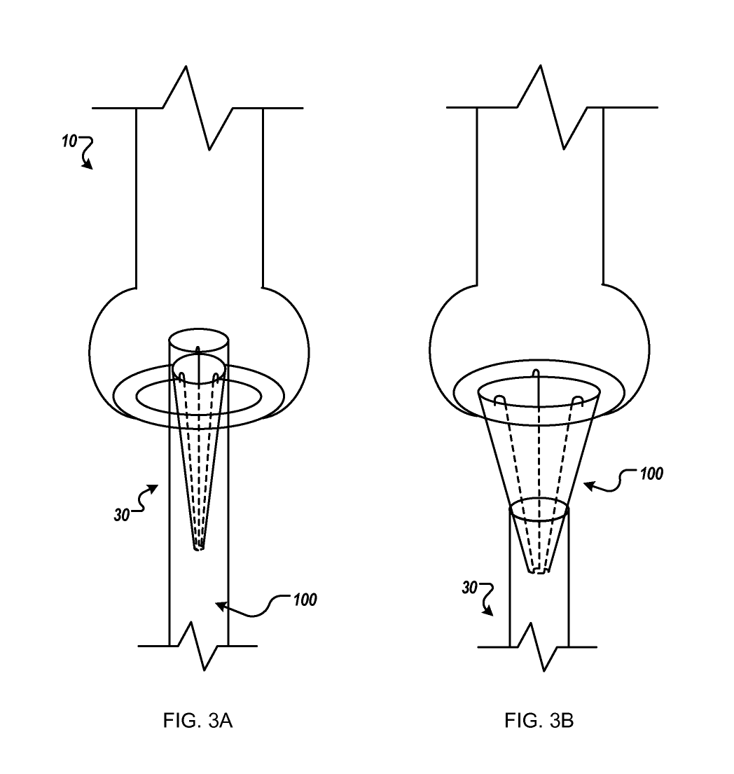

In some embodiments, the membrane 120 is configured such that it can collapse around the strut supports 110. For example, during the diastolic portion of the cardiac cycle, the membrane 120 remains expanded (e.g., the valve 100 is in a closed configuration), thus obstructing fluid flow. During the systolic portion of the cardiac cycle, the membrane material 120 can collapse (e.g., the valve 100 is in a open configuration), thus allowing fluid flow past the valve 100. Near a proximal end 102 of the valve 100, the struts 110 can be configured to engage native tissue to secure the valve 100. For example, near the proximal end 102 of the valve 100, the struts 110 can be configured to attach to native tissue (e.g., native annulus) adhesively, mechanically (e.g., using barbs, clips, hooks, clamps, and the like), chemically, electrically (e.g., “welding”), or using a combination of one or more of these attachment methods. Near a distal end 104 of the valve 100, the struts 110 can meet at a small opening 116. The struts 110 can include any rigid biocompatible material (e.g., plastic, metal, ceramic, and the like) or alloy thereof which retains some amount of flexibility to allow for percutaneous (e.g., through a catheter) deployment. For example, the struts 110 can include a shape memory alloy (e.g., Nitinol) that can be collapsed into a catheter during delivery and then assume an open, conical shape after deployment (see FIG. 3). The membrane 120 covering and affixed to the struts 110 can include any biocompatible flexible/compliant material (e.g., polymers, polyethylene, animal pericardium tissue, other biological tissues, and the like). For example, the membrane 120 can include animal pericardial tissue.

The membrane 120 can have proximal and distal reinforcing regions (e.g., bands, coatings, and the like) 122 and 124, respectively, around the proximal end 102 and distal end 104 of the membrane to reduce or eliminate ripping and fraying after long-term use.

The membrane 120 can be affixed to the struts 110 using any suitable attachment means (e.g., adhesives, clips, sutures, clamps, rings, and the like). The membrane 120 can include enough material between struts 110 to allow for collapse of a portion of the membrane 120 toward a central axis 106 of the valve 110 during systole (the open configuration depicted in FIG. 1B) and to expand and conform to the outer rim of native tissue during diastole (the closed configuration depicted in FIG. 1A). In this way, blood can pass by the valve 110 during systole, but is substantially reduced from passage during diastole. When in the closed configuration, the opening 121 at the distal end 104 of the valve 100 can allow a small amount of fluid to pass through the valve 100 (when compared to when the valve 100 is open) to prevent the pooling of fluid in the bottom (e.g., distal end 104) of the valve 100. This small amount of flow can help to reduce or eliminate the formation of blood clots and to wash the inner surface of the valve 100.

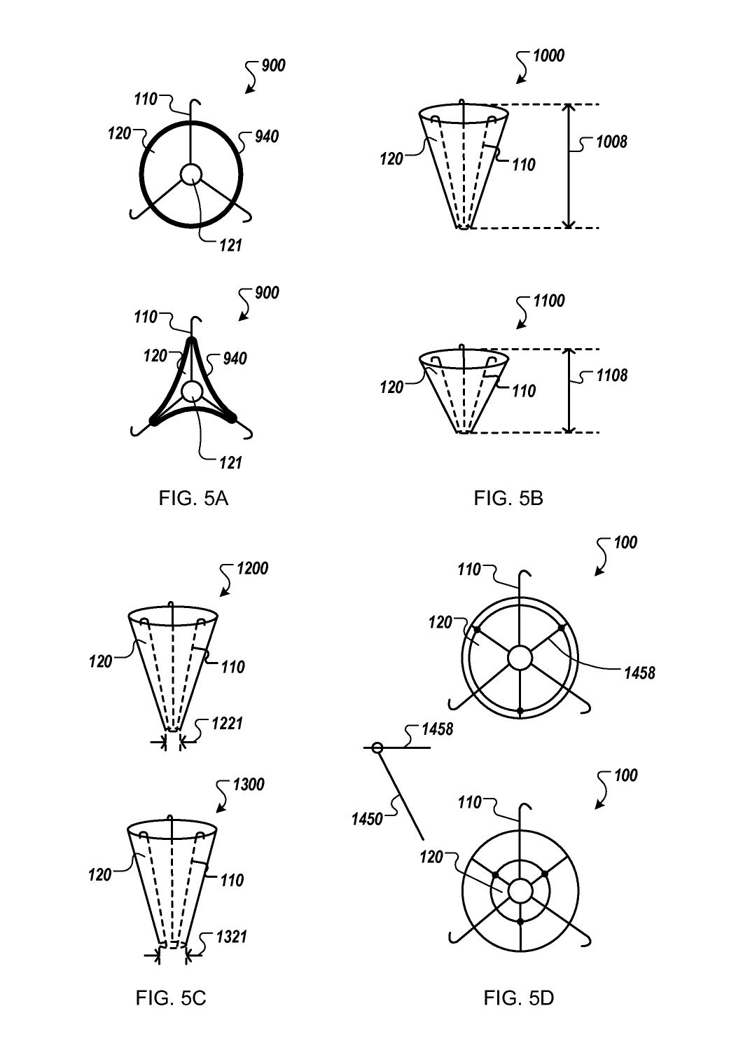

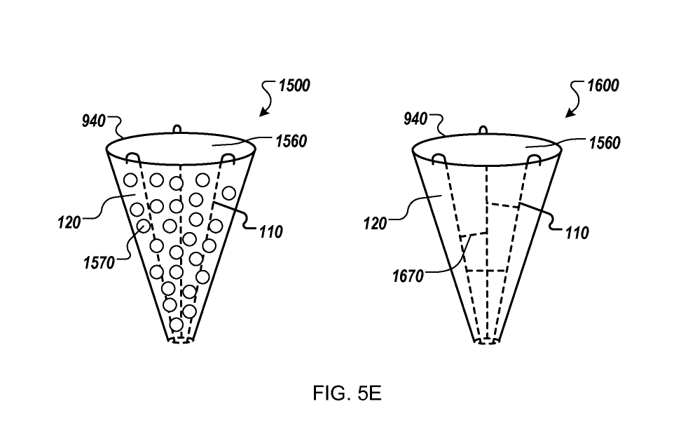

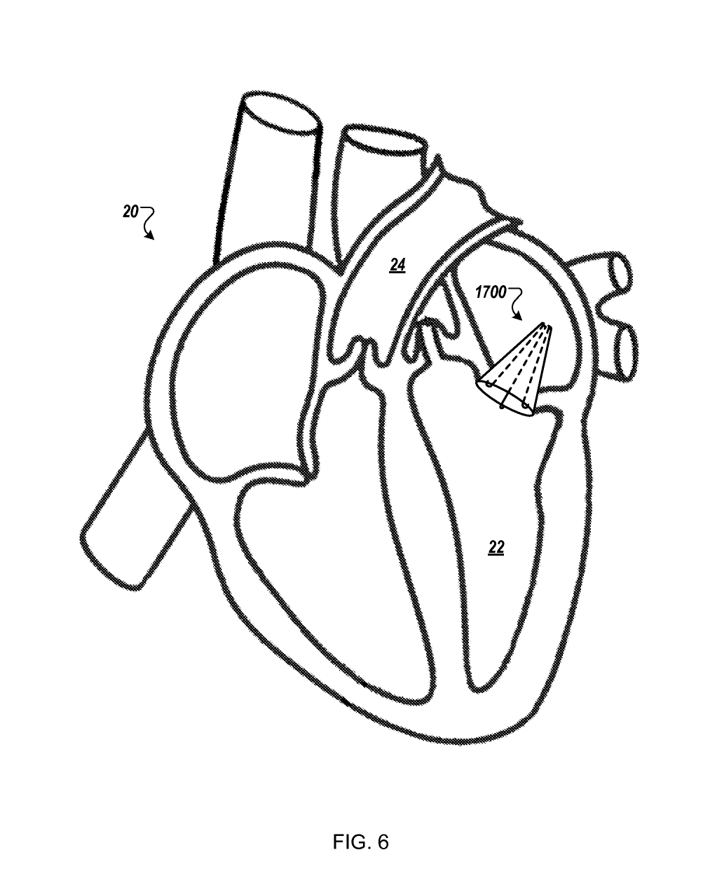

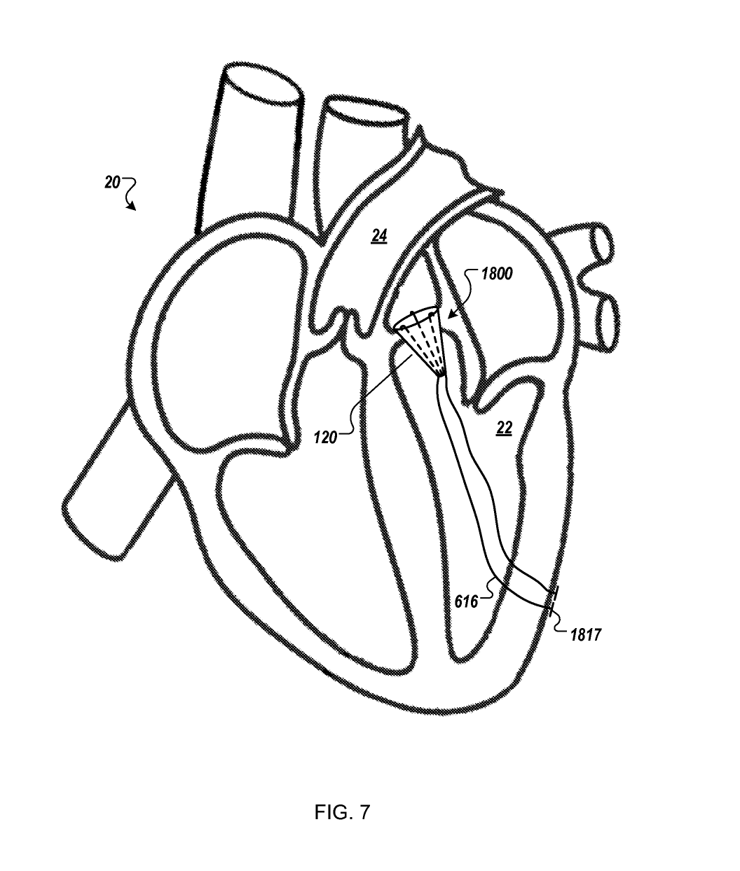

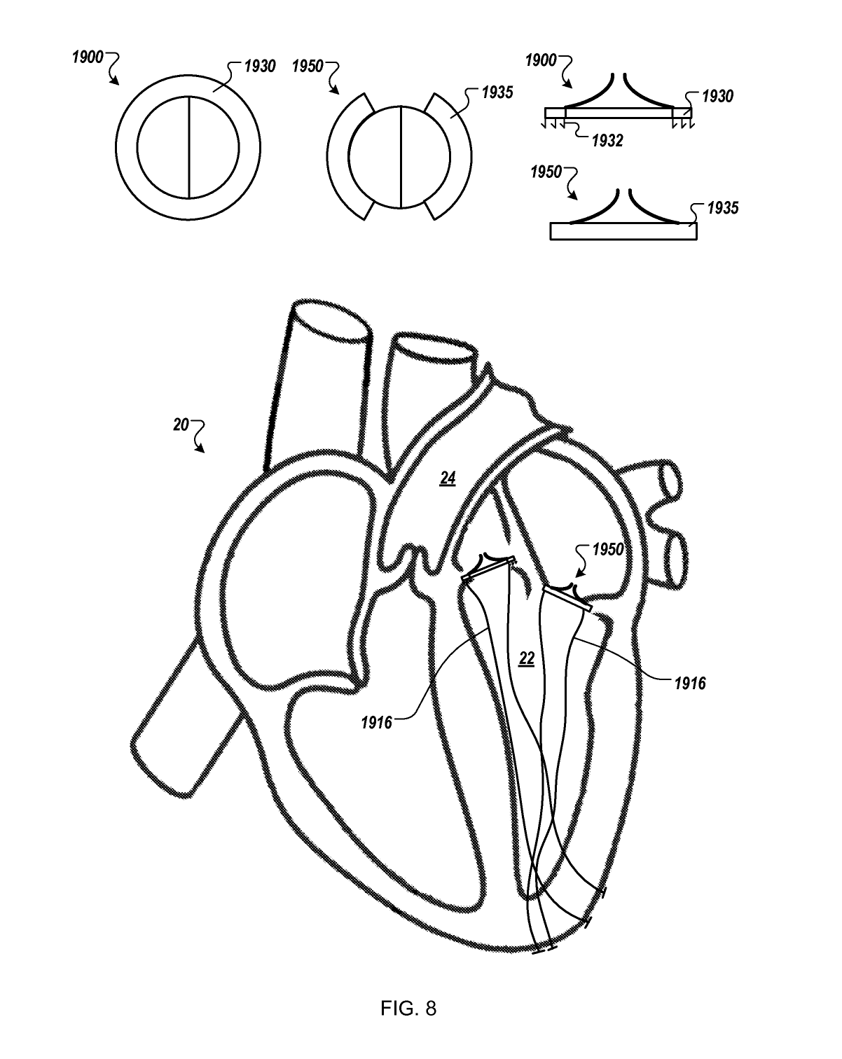

Referring now to FIG. 2, in some embodiments, the valve 100 can be deployed in the heart 20 of a mammal (e.g., a human). For example, the valve 100 can be deployed in the aortic position, thus replacing the native aortic valve. In this configuration, during systole, the contraction of the left ventricle 22 can create a high amount of pressure on the surface of the membrane 120 causing a portion of the membrane 120 to collapse toward the central axis 106 of the valve 110 during systole (see FIG. 1B). This collapse can allow blood to flow around the outer face of the membrane 120 and into the aorta 24. During diastole, the membrane 120 can expand such that the valve 100 transitions back to the closed configuration due to the loss of pressure as the left ventricle 22 relaxes. This configuration can reduce or eliminate blood from flowing from the aorta 24 into the left ventricle 22. In some cases, the transition between the open and closed configurations is assisted with the help of an active component around the proximal rim of the membrane 120 that can bias the valve 100 toward the closed configuration (described in more detail in connection with FIG. 5A).

Referring now to FIGS. 3A-3B, in some embodiments, the valve 100 can be deployed (e.g., minimally-invasively deployed) from a catheter 20 using a transapical approach. The valve 100 can assume a collapsed state when loaded into the deployment catheter 30, as depicted in FIG. 3A. This allows the valve 100 to be inserted into the body through a relatively small opening. In another example, the valve 100 can be positioned via a retrograde aortic approach. In some cases, the deployment of the valve can be via a retrograde aortic approach, a percutaneous/transfemoral approach, or an open surgical approach. Once the deployment catheter 30 is positioned near the native valve annulus, the valve 100 can be pushed out of the catheter, assume its conical shape, and be secured to the native annulus tissue, as depicted in FIG. 3B.

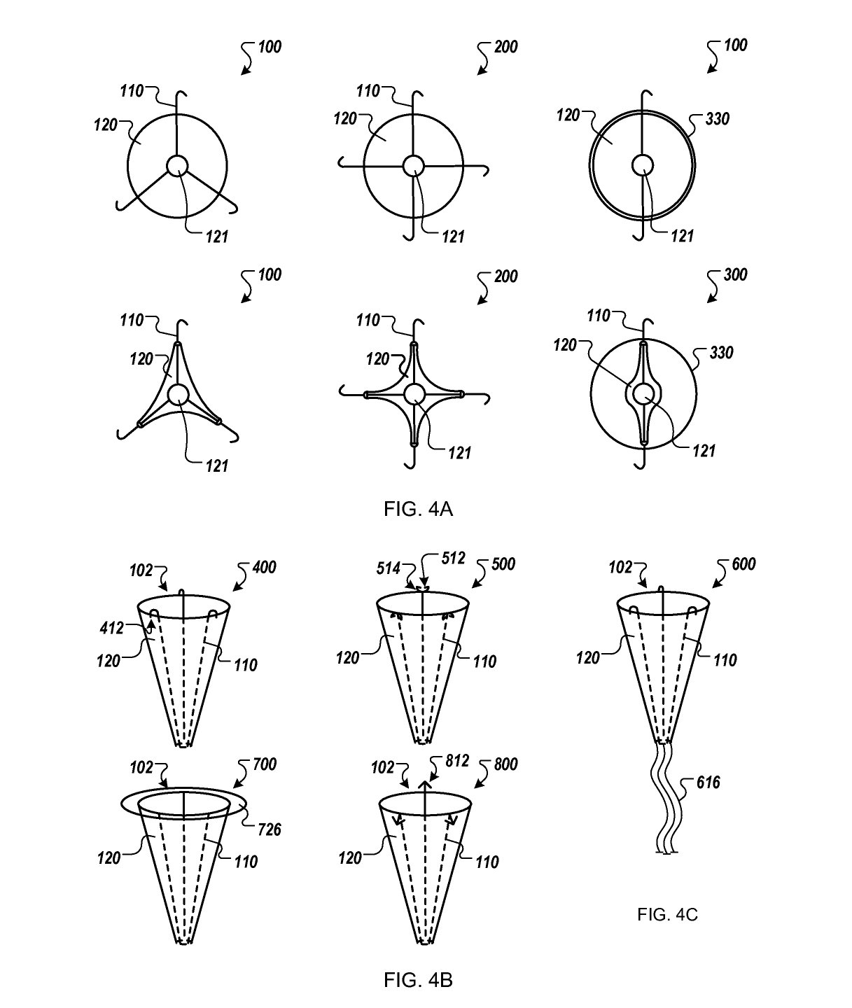

Referring now to FIGS. 4A-4B, in some embodiments, an implantable valve can include one or more of struts 110. For example, as depicted in FIG. 4A, the valves 100, 200, and 300 can include three struts 110, four struts 110, and two struts 110, respectively. In some embodiments, an implantable valve (e.g., the valve 300) can include a support ring 330 that can advantageously provide additional stability for the implantable valve. In some embodiments, an implantable valve can include one or more of a variety of features for permanently securing the valve to native tissue. For example, valve 400 features a design that includes hooks 412 near the proximal end 102 which embed into the valve annulus. In another example, valve 500 features a design that includes barb 512 located on clamps 514 near the proximal end 102 to pinch the annular tissue between each arm. In still another example, valve 600 includes tethering cord anchors 616 which can be used to anchor the valve in distal walls of the heart (see, e.g., FIGS. 4C and 7). In other examples, valve 700 includes a sewing ring 726 whereby the ring 726 can be directly sutured to the annulus, and valve 800 includes barbs 812 near the proximal end 102. In other examples, any type of known anchoring system can be used to secure an implantable valve to native tissue, including adhesives, clamps, staples, barbs, sutures, hooks, screws, and combinations thereof.

Referring now to FIGS. 5A-5E, in some embodiments, an implantable valve can include features advantageous to the implantable valve. For example, FIG. 5A depicts a valve 900 that includes a shape memory ring 940 attached along the outer diameter of the proximal portion of the membrane 120. The shape memory ring 940 can be biased toward an expanded shape to facilitate expanding membrane 120, and thus facilitating the transitioning of the valve to the closed configuration during an obstructive portion of the valve functional cycle (e.g., when the closed valve reduces the flow of material past it). This can advantageously encourage the valve 900 to more quickly transition to the closed configuration, for example, during the short periods of the cardiac cycle. This can, for example, be advantageous when the valve 900 is deployed in the aortic valve position. The shape memory ring 940 can be flexible enough to allow the membrane 120 to deform into the collapsed state at pressures normally occurring during contraction of the left ventricle in systole. In some embodiments, the opposite configuration can be used, for example, in some anatomical positions such as replacing the mitral valve.

Referring to FIG. 5B, in some embodiments, implantable valves can be configured with different lengths 1008 and 1108. For example, FIG. 5B depicts valves 1000 and 1100 with differing lengths. In some embodiments, a valve can be chosen for an application based on length wherein the valve chosen can be based on, for example, the valve that includes a length that facilitates the smallest conformation in a collapsed state in combination with the smallest internal cone volume to reduce fluid pooling. In some cases, the length can be from 15 mm to 50 mm (e.g., 15 mm to 40 mm, 15 mm to 30 mm, 15 mm to 20 mm, 20 mm to 50 mm, 30 mm to 50 mm, or 40 mm to 50 mm). FIG. 5C depicts variations in the distal opening in the cone design to facilitate “washing” of the interior membrane walls and to prevent fluid pooling. For example, valve 1200 includes a smaller diameter opening 1221, while valve 1300 includes a larger diameter opening 1321. In some embodiments, a valve can be chosen such that the diameter of the opening can balance washing and prevention of fluid pooling with prevention of regurgitation of fluid into the wrong cardiac chamber. In some cases, the diameter of the opening can be from 10 mm to 40 mm (e.g., 10 mm to 35 mm, 10 mm to 33 mm, 10 mm to 30 mm, 10 mm to 25 mm, 10 mm to 20 mm, 15 mm to 40 mm, 20 mm to 40 mm, or 30 mm to 40 mm). FIG. 5D depicts a valve 1400 that include rails 1450 attached to a ring structure 1455 on the membrane 120 which allows the membrane 120 to move back and forth from collapsed and expanded orientations. FIG. 5E depicts valves 1500 and 1600 that each include a cover 1560 near the proximal ends 102 of the valves 1500 and 1600. The covers can include materials that are the same or different than material included in the membrane 120. In some embodiments, inclusion of the cover on the valves 1500 and 1600 can restrict the flow of blood into the interior of the valves 1500 and 1600. To facilitate re-expansion during the obstructive portion of a valve cycle, the valves 1500 and 1600 can include the shape memory ring (or other features to encourage expansion of the membrane 120). In some embodiments, the valve 1500 can include other features to encourage expansion of the membrane 120. Expansion features 1570 can include a foam mechanism, a sponge mechanism, a coil mechanism, and the like, within the cone. In some embodiments, the valve 1600 can include mechanical structures 1670 such as springs, coils, shocks, and the like, to encourage expansion of the membrane 120.

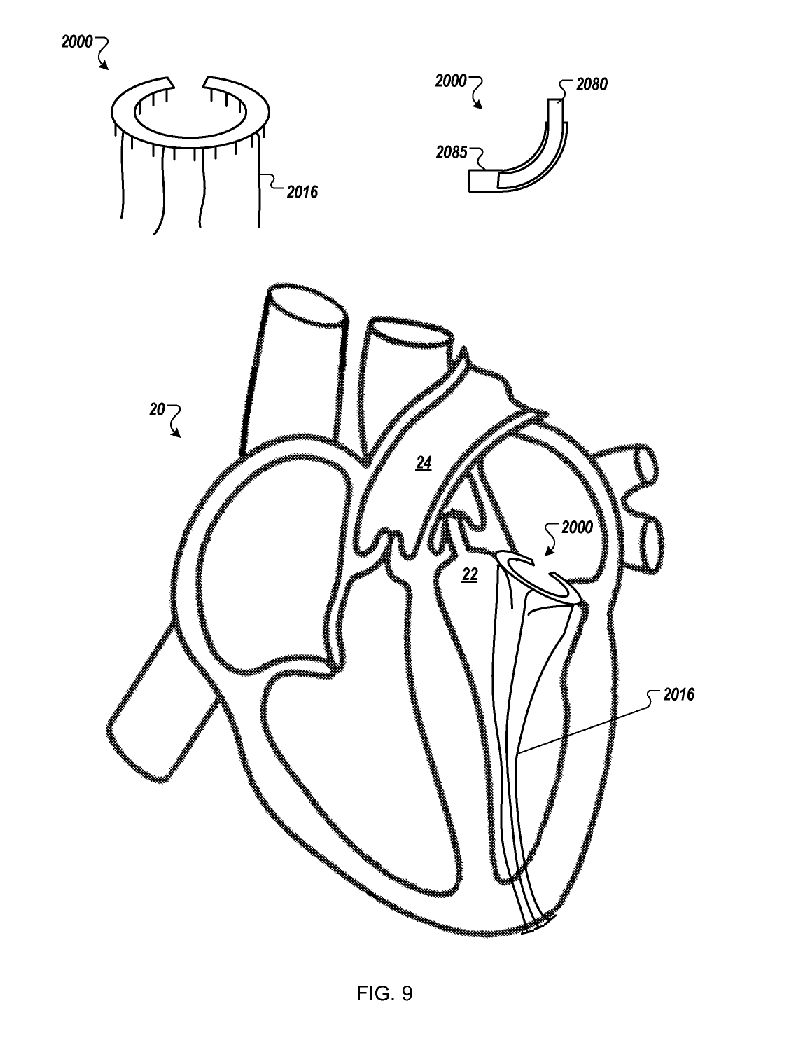

Referring to FIG. 6, in some embodiments, a replacement valve 1700 can be positioned at the mitral position within the heart 20 (e.g., to replace the mitral valve). The valve 1700 can be deployed in the pulmonary or tricuspid position. In the mitral position, the valve 1700 can assume the open configuration (e.g., with the membrane 120 collapsed) during diastole and the closed configuration (e.g., with the membrane 120 expanded) during systole.

Referring now to FIG. 7, in some embodiments, a replacement valve 1800 can include one or more cord anchors 616, which can be used to assist in securing the valve 1800. For example, in FIG. 7, valve 1800 is positioned in the aortic position of heart 20, and cord anchors 616 can be attached to the wall of the left ventricle 22, to the internal papillary muscles of the left ventricle 22, and the like. Cord anchors 616 can include pledgets 1817 or other features to reduce or eliminate the chances of cords 616 pulling through the wall of the heart.

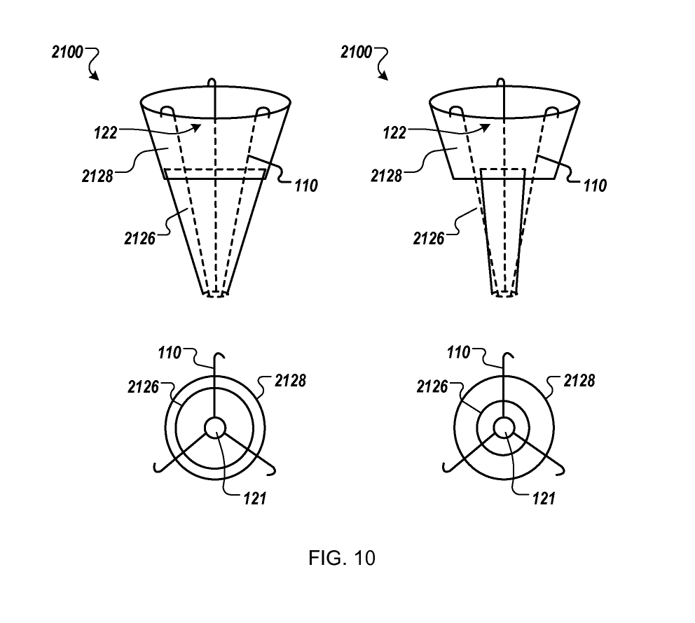

Referring now to FIG. 8, in some embodiments, replacement valves 1900 and 1950 include artificial chordae anchors that can help to maintain valves 1900 and 1950 correctly positioned. Valves 1900 and 1950 can each be placed at an annulus and then cords 1916 (e.g., including sutures, polymers, nylon, and the like) can be run from the valves 1900 and 1950 to the wall of the heart to anchor valves 1900 and 1950 in place. Cords 1916 can be attached to the heart wall, for example, by pledgettes 1917, staples, T-tags, helical screws, suture, and the like. The ability to anchor valves 1900 and 1950 with cords 1917 can allow for very low-profile valve designs. Current artificial valves may rely on stents, cages, and the like to hold the valves in position and anchor to the native annulus. By using cords 1916 attached to the heart, less material may be needed for anchoring at the annulus, and therefore valves 1900 and 1950 can be lower profile. This can make valves 1900 and 1950 easier to deliver and place, less obtrusive, and the like. Cord anchored valve designs (e.g., valves 1900 and 1950) can include one or more of a ring 1930 and a wing 1935 that rests on the top of the native annulus to provide support, with cords 1916 providing anchoring and stability. Ring 1930 and wings 1935 can each rely on radial force/friction or tissue spikes 1932 to provide further anchoring. Cord-anchoring can be used with the valve designs described herein or with any current or traditional valve designs (e.g., bi-leaflet, mechanical, tissue, collapsible, and the like). Cord anchoring can work for valves placed in any location (e.g., aortic, mitral, tricuspid, pulmonary, and the like). Cords 1916 can include an elastic component to allow some give during the cardiac cycle to lengthen and contract, thus minimizing or eliminating the possibility of damage or tearing out. In some embodiments, any number of cords can be used to anchor valves 1900 and 1950 (e.g., one, two, three four, five, or more cords).

Referring now to FIG. 9, in some embodiments, an annuloplasty ring 2000 can include cords 2016 that can assist in anchoring ring 2000. Annuloplasty ring 2000 can be secured to the annulus of the native valve. Ring 2000 can include sliding members 2080 and 2085, which allow ring 2000 to become smaller over time. Anchoring cords 2016 can be used to mechanically slide member 2080 of ring 2000 within member 2085, thereby tightening and shortening ring 2000 over time.

Referring now to FIG. 10, in some embodiments, a valve 2100 can be configured to include a two-piece design. For example, a lower membrane 2126 can include a similar material to membrane 120 described previously (e.g., compliant and collapsible) whereas an upper membrane 2128 can include a more rigid, stiffer composition (e.g., while still being able to have some give to be compliant with biological tissue). Valve 2100 can function in a similar manner to those described in connection with FIGS. 1-6 except that the more rigid upper membrane 2128 may be less likely to collapse during systole, thus continuing to hold its expanded shape. During diastole, rigid upper membrane 2128 can provide a surface 2129 for compliant lower membrane 2126 to meet against, which can reduce or eliminate the amount of blood that can leak back.

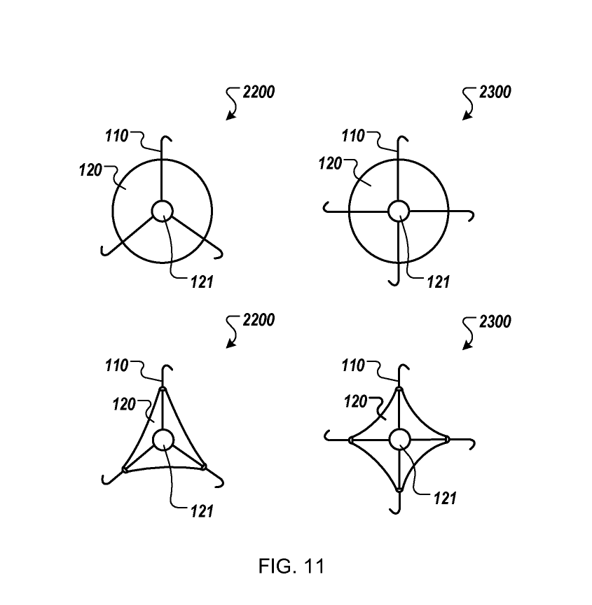

Referring now to FIG. 11, in some embodiments, valves 2200 and 2300 can be configured such that compliant membrane 120 has an overall area such as to only be able to collapse into valves 2200 and 2300 to a certain amount. For example, the amount of compliant material relative to the diameter of the valve (made up by the struts 110) would only allow a certain amount of collapse (e.g., between ⅔ and ¾ of the total circumferential area covered by valves 2200 and 2300). This type of design can balance the ability to allow blood to flow past valves 2200 and 2300 during systole while optimizing closing of the valve in diastole.

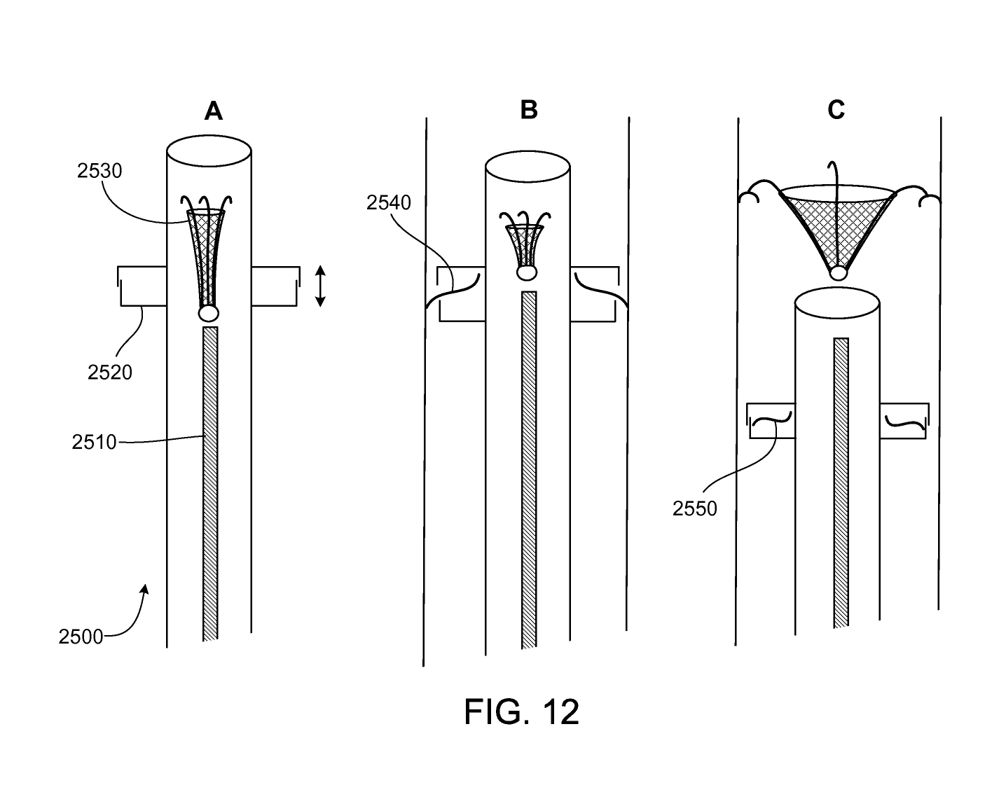

In some cases, a system provided herein can be configured to remove natural valve tissue (e.g., diseased or calcified valve leaflets) and deploy an artificial valve provided herein. With reference to FIGS. 12A-C, system 2500 can include deployment device 2510 configured to deploy valve 2530. In some cases, system 2500 can be configured to include native valve excision device 2520. Excision device 2520 can be configured to have blades that are capable of opening and closing, thereby providing the ability to cut or remove native valve tissue 2540. For example, excision device 2520 can have two blades that can actuate back and forth, thereby having the ability to cut the native leaflets to remove them from, e.g., the valve annulus. The blade movement can be mechanically controlled by a handle (force provided by the surgeon) or by hydraulic pressure (fluid, gas, etc.) in order to provide a quick, forceful cutting motion to remove the leaflets (e.g., diseased leaflets). After the leaflets are cut, the device can be pulled back while simultaneously advancing the new valve forward with the deployment tool. In this way, there is a nearly immediate replacement of the native valve with a new valve. In some cases, the removed native valve tissue 2550 can be retained in the system as it is being removed from the patient.

A number of embodiments of the invention have been described. Nevertheless, it will be understood that various modifications may be made without departing from the spirit and scope of the invention. Accordingly, other embodiments are within the scope of the following claims.

What is claimed is:

1. An artificial heart valve for placement within a mammal, wherein said heart valve comprises:
(a) at least two struts having a proximal portion and a distal portion, wherein said proximal portion is configured to attach to heart tissue, and wherein said struts, when said heart valve is placed within said mammal, converge towards an axis in a direction from said proximal portion to said distal portion, and
(b) a membrane structure attached to said struts and configured to form a wall around said axis, wherein at least a portion of said membrane structure is capable of expanding and collapsing movement, wherein during said expanding movement said portion of said membrane structure moves away from said axis to form a closed position of said heart valve, wherein during said collapsing movement said portion of said membrane structure moves toward said axis to form an opened position of said heart valve,
wherein, when said heart valve is placed within said mammal and in said opened position, blood upstream of said heart valve is capable of moving past said heart valve between said membrane structure and the mammal's heart tissue, and wherein, when said heart valve is placed within said mammal and in said closed position, movement of blood upstream of said heart valve past said heart valve between said membrane structure and the mammal's heart tissue is limited.

2. The heart valve of claim 1, wherein said mammal is a human.

3. The heart valve of claim 1, wherein said proximal portion of said struts is configured to attach to heart tissue via an adhesive, clamp, staple, barb, suture, hook, screw, or combination thereof.

4. The heart valve of claim 1, wherein said membrane structure comprises flexible biocompatible material.

5. The heart valve of claim 1, wherein said membrane structure comprises a polymer.

6. The heart valve of claim 1, wherein said membrane structure comprises animal pericardium tissue.

7. The heart valve of claim 1, wherein said membrane structure forms a conical shape.

8. The heart valve of claim 1, wherein said membrane structure forms a conical shape defining a lumen comprising an opening at first end and an opening at a second end, wherein the opening at said first end is larger than the opening at said second end.

9. The heart valve of claim 1, wherein said heart valve comprises a first end defining a diameter and a second end defining a diameter, wherein the diameter of said first end is larger than the diameter of the second end, and wherein said first end defines an opening.

10. The heart valve of claim 9, wherein said second end defines an opening, wherein the opening of said second is smaller than the opening at said first end.

11. The heart valve of claim 1, wherein said struts comprise flexible material.

12. The heart valve of claim 1, wherein said struts comprises a shape memory material.

13. The heart valve of claim 12, wherein said shape memory material is nitinol.

14. The heart valve of claim 1, wherein said heart valve is capable of being placed within said mammal percutaneously.

15. The heart valve of claim 1, wherein said heart valve is capable of moving from a collapsed position during delivery to said mammal to an expanded position after placement within said mammal.

16. The heart valve of claim 1, wherein said heart valve comprises a ring structure attached to said proximal portion of said struts.

17. The heart valve of claim 16, wherein, when said heart valve is placed within said mammal and in said opened position, blood upstream of said heart valve is capable of moving past said heart valve between said membrane structure and said ring structure, and wherein, when said heart valve is placed within said mammal and in said closed position, movement of blood upstream of said heart valve past said heart valve between said membrane structure and said ring structure is limited.

18. The heart valve of claim 16, wherein said ring structure comprises a shape memory material biased to promote movement of said proximal portion of said struts away from said axis during placement of said heart valve within said mammal.

19. The heart valve of claim 1, wherein said heart valve comprises a ring structure attached to said distal portion of said struts.

20. The heart valve of claim 1, wherein said heart valve comprises a tethering cord anchor.

21. The heart valve of claim 20, wherein said tethering cord anchor is configured to extend from said heart valve and across at least a portion of the heart chamber downstream of said heart valve when said heart valve is placed within said mammal.

22. The heart valve of claim 21, wherein said tethering cord anchor comprises an adhesive, clamp, staple, barb, suture, hook, screw, or combination thereof configured to attach said tethering cord anchor to a wall of said heart chamber.
